(19) United States
(12) Patent Application Publication
(10) Pub. No.: US 2020/0269072 A1
(43) Pub. Date: Aug. 27, 2020

(54) Device And Method For Damaging Parasites Using Ultrasonic Reflection

(71) Applicant: PARASONIC LTD., Tel-Aviv (IL)

(72) Inventors: Mor Miri Cohen, Iryat Bialik (IL); Jonathan Yalom, Tel-Aviv (IL); Michael Kardosh, Kiryat Ono (IL)

(21) Appl. No.: 16/067,957

(22) PCT Filed: Jan. 5, 2017
(86) PCT No.: PCT/IL2017/050018

§ 371 Date: Jul. 3, 2018

Related U.S. Application Data
(60) Provisional application No. 62/275,264, filed on Jan. 6, 2016.

(51) Int. Cl. A61N 7/00; A46B 15/00

(52) U.S. Cl. CPC A61N 7/00; A46B 15/0028; A46B 2200/104; A61N 2007/0069; A46B 2200/1093; A61N 2007/0034

(57) ABSTRACT

A device for damaging a parasite while being on a surface of a body of a mammal. The device is a hand held device with an ultrasound transmitter for generating ultrasound radiation and a plurality of teeth extending from a base of the device and arranged alternately.

### BACKGROUND

Head lice infestation, also known as pediculosis capitis, is a well-known epidemic and a common problem in pediatric practice. Domestic pets such as dogs and cats and farm animals may suffer from parasites, such as nits and ticks. Most commonly used methods for removing parasites such as lice from human hair or parasites from pets or other animals include the use of neurotoxic topical agents. Due to the toxicity of these agents, there is a constant drive to find other parasite removers, for example, non-neurotoxic topical agents, plant-based compounds and natural oils. For lice treatment in humans there are also oral treatment and physical methods. Pets or farm animals are sometimes treated with injections or materials that are dissolved into the skin of the animal.

The major concern regarding the efficiency of each of the methods is the ability of the parasite to develop resistance to the various (in particularly chemical) treatments. The only methods that the parasite cannot develop resistance are the physical methods. It may be beneficiary to develop a simple and cheap physical method for damaging parasites in mammals' hair.

### SUMMARY

Embodiments of the invention provide a method and a device for damaging a parasite while being on a surface of a body of a mammal. The device may be a hand held device which comprises an ultrasound transmitter for generating ultrasound radiation and a plurality of teeth extending from a base of the device and arranged alternately. It will be noted that alternating arrangement of teeth may refer to single type of tooth each alter, tow teeth of one type alternating with one tooth of another type, etc. The plurality of teeth comprises a plurality of vibrating teeth and a plurality of reflecting teeth, each having a reflecting chamber. It will be noted that plurality of teeth refers to one pair of teeth, each of another type, or mote. A reflecting chamber may be made of metal such as stainless steel, or be a gas-filled volume, or gas enveloped in a thin plastic skin, thinner than ¼ wavelength.

The ultrasound radiation transmitted from each of the vibrating teeth towards each of the reflecting teeth and may be reflected back by a reflecting tooth itself or by a reflecting chamber of the reflecting tooth which allows damaging a parasite located between the vibrating tooth and the reflecting tooth. The reflecting tooth made of reflecting material or having a chamber that may be filled with gas or other reflecting material.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 is a schematic illustration of an exemplary system for damaging parasites according to some embodiments of the invention;

FIG. 2 is a schematic illustration of the arrangement of the teeth in a system for damaging parasites according to some embodiments of the invention;

FIG. 3A is a schematic illustration of adjacent teeth of a system for damaging parasites according to some embodiments of the invention;

FIG. 3B is a schematic illustration of a reflecting tooth of a system for damaging parasites according to some embodiments of the invention;

FIG. 4 is an exemplary diagram representing an acoustic field in an ultrasonic gel layer according to some embodiments of the invention;

FIG. 5A is a top view of a hand held device according to embodiments of the invention;

FIG. 5B is a bottom view of a hand held device according to embodiments of the invention;

FIG. 5C is an illustration of a louse and a louse egg before and after treatment according to embodiments of the present invention

FIG. 6A is a schematic illustration of an exemplary row of teeth of a device for damaging parasites according to some embodiments of the invention;

FIG. 6B is a schematic illustration of an elastic balloon according to some embodiments of the invention;

FIG. 6C is a schematic illustration of a piezoelectric balloon according to some embodiments of the invention; and

FIG. 6D is a schematic illustration of a piezoelectric balloon according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Embodiments of the invention may allow damaging or destruction of parasites and parasite's eggs by ultrasound radiation reflected from a reflective tooth. A hand held device is placed on a surface of a body of a mammal, such as a human or an animal and is configured to transmit ultrasound radiation from a first tooth of the device towards a second tooth having a reflecting material or chamber, When a parasite is located between the first tooth and the second tooth it may be hit by the ultrasound energy applied from the first tooth and at least by a portion of the energy which is reflected back from the reflective chamber.

Using a reflective chamber may allow using lower level of ultrasound radiation which is desirable especially when treating human or other mammal skin. Low levels of ultrasound radiation which are required with embodiments of the invention, may allow use of smaller power source and hence smaller batteries. An exemplary intensity may reach 0.07 Watt/cm2, however, any other intensity may be used with embodiments of the invention.

The device, system and method described in embodiments of the invention may treat a variety of parasites and parasites' eggs. Exemplary parasites that may be treated may include, but are not limited to, lice, fleas, nits, insects and ticks. Embodiments of the invention may include a hand held device configured to apply ultrasound radiation which may be reflected by a reflective tooth or a reflective chamber such that the ultrasound energy level hitting a parasite treated by the device may be higher than the original level of radiation generated. As used herein, the term “damage” refers to any process which reduces the viability of a parasite, harms the parasite such as to prevent the parasite from moving, reproducing, hatching (e.g., parasite eggs) or the like. In some embodiments, the ultrasound radiation is preferably selected to kill the parasite.

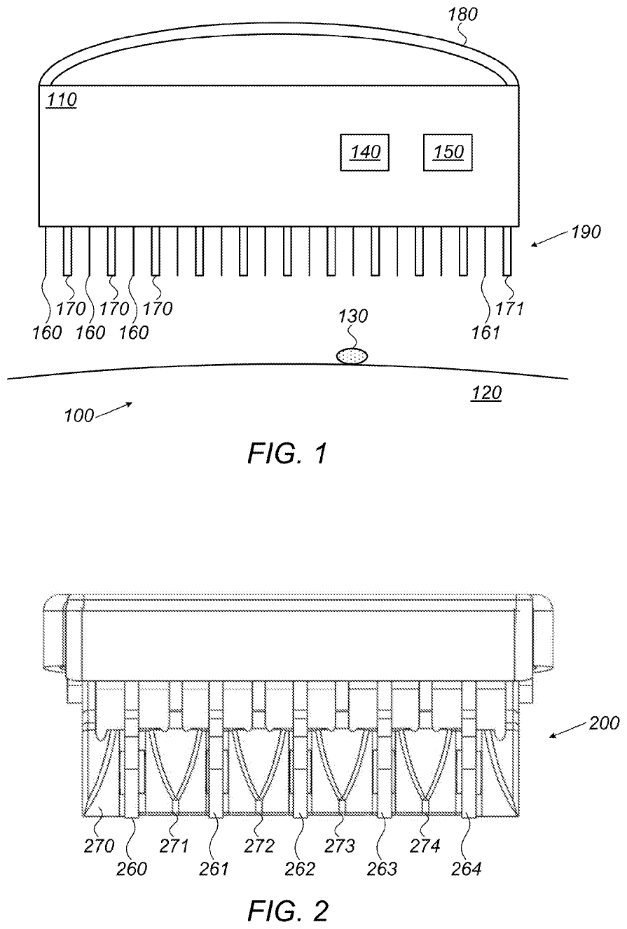

Reference is now made to FIG. 1 which is a schematic illustration of an exemplary system 100 for damaging parasites according to some embodiments of the invention. System 100 may include a hand held device 110 useable to damage a parasite 130 while being on a surface 120 of a body of a mammal, on hair or on fur. Hand held device 110 may include a knob, a handle or a grip 180, a transmitter circuitry 150 for generating ultrasound radiation and a power source 140 for supplying power to transmitter circuitry 150. Hand held device 110 may further include a plurality of teeth 190 extending from the body of hand held device 110. Teeth 190 may include a plurality of teeth from two or more types. A first type of teeth 170, referred to herein as “piezoelectric teeth” or “vibrating teeth” and a second type of teeth 160, referred to herein as “teeth having a reflective region” or “reflection teeth”.

Device 110 is preferably a mobile device which may be powered by an independent power source. For example, power source 140 may be or may include a battery; alternatively, power source 140 may include a power cord for connecting device 110 to an external power source. Electronic circuitry 150 may be configured to control the generation of the ultrasound signals by delivering electronic signals to each one of piezoelectric teeth 170. Electronic circuitry 155 may include, for example, a Phase Locked Loop (PLL) system to control the ultrasonic frequency, one or more amplifiers and one or more electronic circuits such as a “push-pull power stage” circuit to allow transfer of power to each tooth of piezoelectric teeth 170. In some embodiments of the invention, electronic circuitry 155 may include circuitry to generate a fixed predetermined frequency.

In some embodiments of the invention a vibrating element of an ultrasound transmitter may be mounted on the body of hand held device 110 so as to collectively vibrate teeth 170 while in other embodiments of the invention, optionally, each tooth of teeth 170 may vibrate by means of one or more vibrating elements mounted next to each respective tooth adjacent the teeth row or between teeth aligned in the row. According to some embodiments of the invention each tooth of teeth 170 may include piezoelectric material while in other embodiments ultrasound transducer may be located onside teeth 170, aside of the row of teeth 170 in proximity to teeth 170, in front of teeth 170, above teeth 170 or in other location which may allow each tooth of teeth 170 to transmit and/or generate ultrasound radiation.

In some embodiments of the invention hand-held device 110 may be placed on or at proximity to surface 120 so as to deliver ultrasound radiation to parasite 130. A parasite being located between a vibrating tooth and a reflecting tooth, e.g., a piezoelectric tooth 171 and a reflecting tooth 161 may be radiated with the ultrasonic radiation radiated from piezoelectric tooth 171 as well as with at least a portion of the transmitted radiation being reflected by the reflecting tooth itself or by the reflecting area or region of reflecting tooth 161 as described hereinafter in embodiments of the invention. The transmitted ultrasound energy is thus directed at a parasite from two opposing directions, nearly doubling the applied energy, thereby resulting in increased damage to tissues of parasite 130.

Teeth 190 may be arranged in an array of one or more rows. In some embodiments of the invention each row may include piezoelectric teeth 170 and reflecting teeth 160 arranged alternately. Each vibrating tooth may reside between two reflecting teeth and each reflecting tooth may reside between two piezoelectric teeth, except for the first tooth and the last tooth in the row. Such an arrangement having a reflecting tooth on each side of each piezoelectric tooth may allow reflection of the radiation arriving from the piezoelectric tooth from two adjacent reflecting teeth, a first on the right side of the piezoelectric tooth and a second on the left side of the piezoelectric tooth. It should be understood that in other embodiments of the invention the arrangement of teeth may be different, for example, teeth 170 may be reflecting teeth and teeth 160 may be piezoelectric teeth or both teeth 160 and 170 may be piezoelectric teeth. Any other arrangement of teeth may be used.

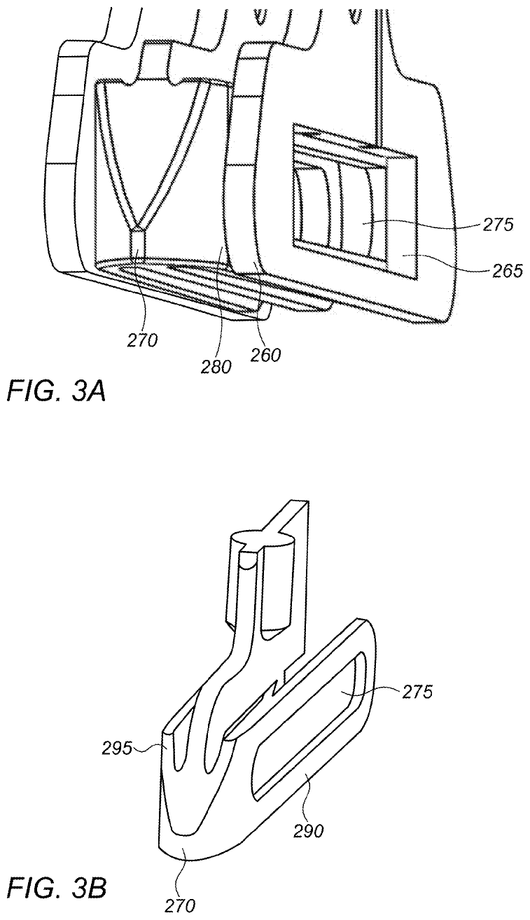

Reference is made now to FIG. 2 which is a schematic illustration of the arrangement of the teeth in a system for damaging parasites according to some embodiments of the invention. Hand held device 110 of FIG. 1, may include a plurality of rows, each row includes a plurality of teeth. An exemplary row of teeth 200 is shown in FIG. 2. Row 200 may include a plurality of reflecting teeth 270, 271, 272, 273 and 274 and a plurality of piezoelectric teeth 260, 261, 262, 263 and 264. The reflecting teeth and piezoelectric teeth are arranged alternately, such that each piezoelectric tooth is placed or located between two reflecting teeth and each reflecting tooth is placed or located between two reflecting teeth. For example, piezoelectric tooth 260 is placed, positioned or located between reflecting teeth 270 and 271 and reflecting tooth 272 is placed, positioned or located between reflecting teeth 261 and 262. It should be clear that the number of teeth shown in FIG. 2 is an exemplary number and any number of teeth may be used. Although embodiments of the invention are not limited in this respect, row 200 may start with a reflecting tooth.

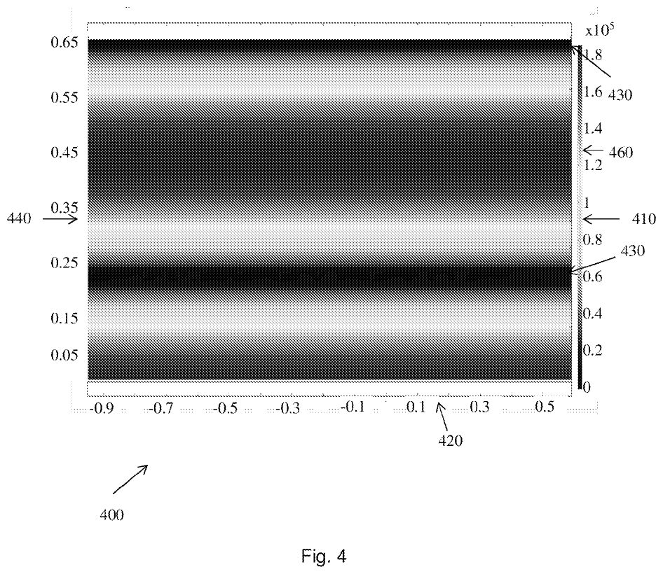

FIG. 3A is a schematic illustration of adjacent or neighboring teeth of a system for damaging parasites according to some embodiments of the invention. A tooth 270 also referred to herein as “reflecting tooth” and an adjacent piezoelectric or vibrating tooth 260 are shown in FIG. 3A. Each piezoelectric tooth, e.g., tooth 260, may radiate ultrasonic radiation. In some embodiments of the invention, tooth 260 may comprise piezoelectric material such as, for example, a crystal or a capacitive micromachined ultrasonic transducers (CMUT) which may act as an ultrasonic transducer while in other embodiments the piezoelectric material may be located elsewhere, e.g., at a central location in the hand held device, above every piezoelectric tooth or next to every piezoelectric tooth. In some embodiments of the invention, tooth 260 may include a hole, a space, a gap or a chamber 265 for placing the piezoelectric material. It should be clear to a person skilled in the art that the piezoelectric material may be located at any location such as to allow ultrasonic radiation to be transferred from each of the piezoelectric teeth, preferably and mainly towards its neighboring reflecting teeth.

In some embodiments, each reflecting tooth, e.g., tooth 270, may be made from or may include a reflective material, such as for example, stainless still, metal, Styrofoam or the like while in other embodiments, each reflecting tooth, e.g., tooth 270, may include a reflective region, 275 which may be implemented by a hole, a chamber, cavity, a gap or a space in the body of tooth 270. Region 275 may include gas or another reflecting material such as, gas, metal and any other material to reflect ultrasonic radiation. A space 280 is created between any pair of adjacent reflecting tooth and piezoelectric tooth, e.g., reflecting tooth 270 and piezoelectric tooth 260. According to embodiments of the invention, a parasite which is placed between a piezoelectric tooth and an adjacent reflecting tooth, for example, at space 280, may be radiated with ultrasonic energy from a piezoelectric tooth, e.g., tooth 260 and with at least a portion of the reflected ultrasonic energy arriving from the reflective chamber of the reflecting tooth, e.g., from reflective chamber 275 of tooth 270. Due to the change in acoustic impedance, ultrasound waves which reach the subject at chamber 275, e.g. gas, are reflected. In some embodiments, the gas or air in reflective region serves as a reflector for the ultrasound energy. In some embodiments of the invention, a parasite which is placed between a piezoelectric tooth and an adjacent reflecting tooth may be radiated with ultrasonic energy from the piezoelectric tooth and with reflected ultrasonic energy arriving from two reflective chambers of two reflecting teeth on both sides of the piezoelectric tooth.

Reference is made now to FIG. 3B which is a schematic illustration of a reflecting tooth of a system for damaging parasites according to some embodiments of the invention. In some embodiments, tooth 270, may be made from a reflective material, such as for example, stainless still, metal or the like. In other embodiments, tooth 270, may be made from a non-reflective material such as for example, plastic which absorbs the acoustic wave transmitted from the piezoelectric tooth, for example, from piezoelectric tooth 260, and prevents its reflection. Tooth 270 may include a reflective chamber 275 which may include a material reflective to ultrasonic radiation, such as for example, gas, air or metal. In some embodiments of the invention, reflective chamber 275 may be a hole, gap, slit or opening in tooth 270 such that the reflection of the ultrasonic radiation is achieved due to the gas residing within the hole, in tooth 270. In some embodiments, reflective chamber 275 may be covered with an acoustically transparent material with a low acoustic attenuation which does not affect or influence the propagation of ultrasonic radiation such as a nylon sheet or an adhesive sheet such that the surface of tooth 270 may remain flat and smooth. In some embodiments chamber 275 may include gas captured, enclosed or sealed within an acoustically transparent material such that the acoustic wave arriving from piezoelectric tooth 260 may go via a parasite, possibly passing through the parasite first then to the transparent material and then being reflected by the captured gas. Alternatively, the material may not be transparent, but rather absorbing, yet very thin, so as not to reduce the acoustic power reflected from the complete interaction between the material and backing gas. Tooth 270 may include two symmetric and/or identical wings 290 and 295 which may flexibly be connected to the body of tooth 270 to allow movement towards each other while being pressured, for example, when a hair or a parasite is placed between tooth 260 and tooth 270. According to embodiments of the invention, wings 290 and 295 may be made from an elastic material to allow such a movement.

Embodiments of the invention may allow one or more parasites such as lice, fleas or nits to reside between a radiating surface, e.g., the surface of the piezoelectric tooth facing an adjacent reflective tooth, and a reflective surface, e.g., reflective chamber 275 of tooth 270 facing the adjacent radiating tooth. The acoustic radiation transmitted from the radiating surface may be reflected back, at least partially, by the substance—gas or other reflecting material—of reflective chamber 275 so as to allow a creation of a standing acoustic wave/s between the reflective chamber and the surface of the piezoelectric tooth. Such a standing acoustic wave may be desired to produce high ultrasonic intensity between two adjacent tooth, in order to damage tissues of a parasite residing between the reflective chamber and the surface of the piezoelectric tooth as the parasite may be hit from both the acoustic radiation transmitted from the radiating surface and by at least a portion of the acoustic radiation reflected back from the reflective chamber.

In some embodiments of the invention, the surface of the piezoelectric tooth may be covered by ultrasonic gel layer or other lotion. For example, a gel layer with thickness of ¾ λg wherein λg is the wavelength of the acoustic radiation within the ultrasonic gel, e.g., arriving from a vibrating tooth and measured at a resonance frequency (Fr) of, for example, 1.60 MHz-1.68 MHz. Other frequency may be set. The relations between λg, Fr and Cg which is the sound velocity in the ultrasonic gel are defined in the following equation:

μg=Cg/Fr (1)

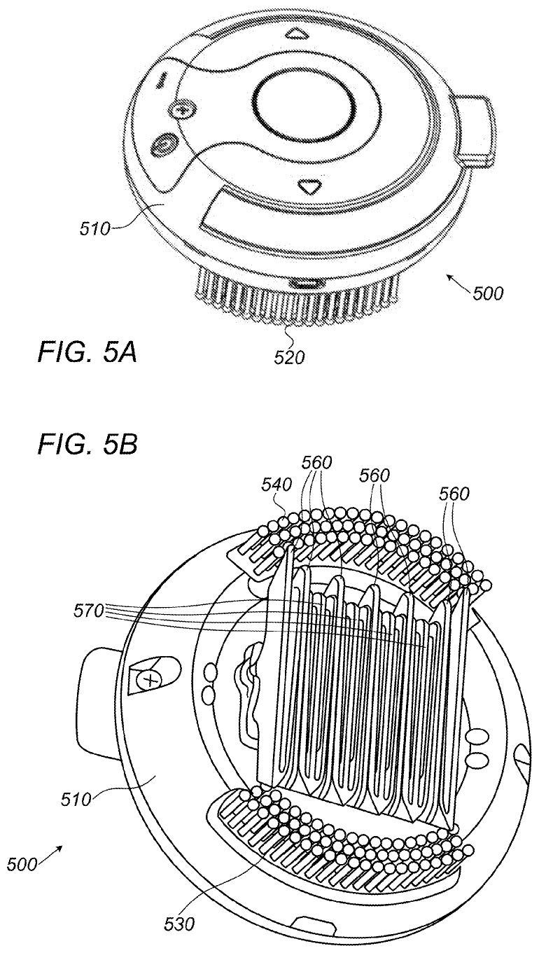

In some embodiments, a standing acoustic wave is formed between a surface of the piezoelectric tooth 260 and the reflective chamber 275, the standing acoustic wave may have at least two maximum values, a first maximum value may be measured at the surface of the piezoelectric tooth and a second maximum value may be measured at a distance of ½ λg from the surface of the piezoelectric tooth. Reference is made now to FIG. 4 which is an exemplary diagram representing an acoustic field in an ultrasonic gel layer according to some embodiments of the invention. Diagram 400 shows the acoustic field in an ultrasonic gel layer covering, for example, a surface of a piezoelectric tooth of a system or a device for damaging parasites of FIG. 1. A right vertical scale 410 represents pressure units as a color scale while the left vertical axis 440 of diagram 400 represents the gel thickness above the surface of the vibrating tooth. Diagram 400 represents the pressure in an ultrasonic gel layer having thickness of 0.65 millimeters with an acoustic wave frequency of 1.64 Mega Hertz (MHz). In exemplary diagram 400, ½ λg equals 0.4 millimeters (mm) which is substantially the distance between the surface of a piezoelectric tooth and the reflecting tooth. The minimum value of the acoustic field, marked with numeral 430, is at 0.2 mm and at 0.6 mm from the surface of a piezoelectric tooth with 1.2*10\hat{ }5 pascal (Pa) and the maximum value of the acoustic field, marked with numeral 460, is at 1.8*10\hat{ }5 Pa and 0.03 mm and 0.4 mm at the surface of a piezoelectric tooth itself.

Although embodiments of the invention are not limited in this respect, the ultrasonic radiation being used in some embodiments of the invention may be a “non focused” ultrasonic energy which may not be focused by any lens. For example, a single piezoelectric tooth or a piezoelectric transducer may generate amplitude of 10 Watt for duration of 2 milliseconds.

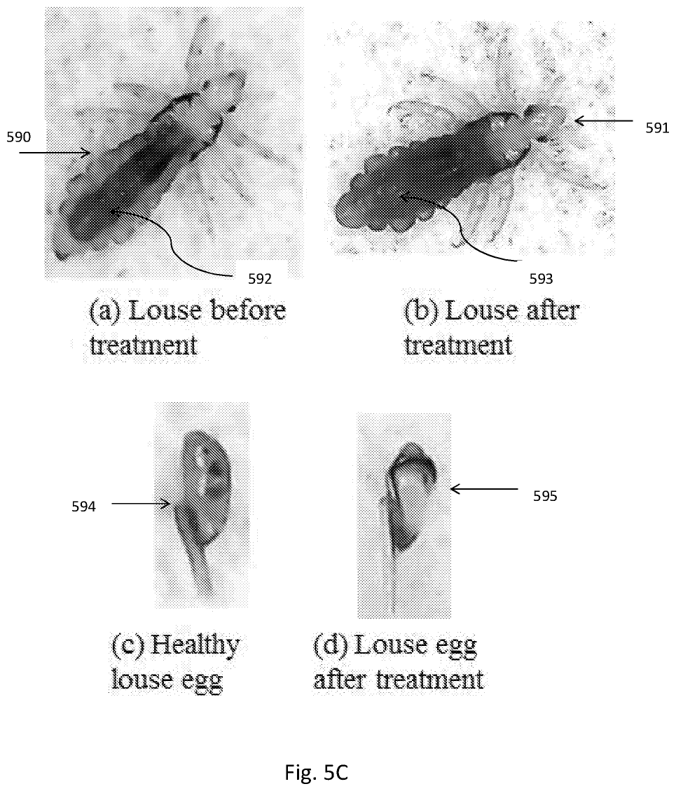

Reference is now made to FIG. 5A which is a top view isometric of hand held device 500 according to embodiments of the invention, and to FIG. 5B which is a bottom view of a hand held device according to embodiments of the invention. Embodiments of the invention may include a plurality configurations, shapes, structures and arrangements of a hand held device, e.g, a hand held device 110 of system 100 of FIG. 1. In the exemplary configurations of FIGS. 5A and 5B hand held device 500 may have a round shape body 510, however, it should be clear that the shape of body 510 is an exemplary shape and other external boundaries, outlines, or external surface shapes may be used in embodiments of the invention.

Hand held device 500 may include a plurality of teeth 520 extending from body 510. Teeth 520 may resemble comb teeth used for combing or brushing hair of a human or a mammal In the exemplary shape of hand held device 500 teeth 520 may be arranged, for example, in two identical groups of teeth 540 and 530 positioned around portions of the perimeter of body 510 as shown in

FIG. 5B. Teeth 520 may be particularly useful when hand held device 500 is operated for damaging a parasite residing on a hairy or furry surface of a mammal as the damaging by ultrasound radiation is accompanied by hair or fur combing while facilitating the damaging of the lice and/or nits from the hair or fur.

Reference is made back to FIG. 5B. Hand held device 500 may include a plurality of piezoelectric teeth 560 and a plurality of reflecting or vibrating teeth 570 arranged alternately with each other and useable for damaging head lice and/or nits. In some embodiments of the present invention the spacing or gaps between every two adjacent teeth is selected to allow passage of a hair shaft, but may prevent passage of lice or nits. The spacing between every two adjacent teeth 570 and 560 may vary, for example, from 0 mm to 10 mm, however, any spacing between two adjacent teeth 570 and 560 may be used.

Embodiments of the invention may allow reducing a viability of a parasite or an egg of a parasite, e.g., a louse or a louse egg, by providing ultrasonic radiation and reflection of ultrasonic radiation. Lice and louse eggs have viable tissues and when hit by a sufficient amount of ultrasonic radiation may cause damage of internal membranes of the tissues of both lice and louse eggs. A membrane of an internal organ of a louse or a louse egg may be damaged, hurt or torn due to the ultrasound radiation provided by a vibrating tooth and by at least a portion of the ultrasound radiation reflected back from a reflecting region in an adjacent reflecting tooth. When a tissue membrane is hurt or torn due to the ultrasound radiation, blood may be diffused out of the hurt organ, membrane or tissue toward other areas of a louse body or a louse egg.

Reference is made now to FIG. 5c which shows an illustration of a louse and a louse egg before and after being treated according to embodiments of the present invention. The difference between a louse 590 before treatment and a louse 591 after treatment may be visualized by the difference between the size of dark areas, which represent areas with blood. Before being radiated with ultrasound radiation and its reflection, a dark area 592 representing blood in non-treated louse 590 is located at a central defined area of louse 590 body, e.g., an organ such as a digestive tract of the louse. A dark area 593 representing blood in a louse 590, after being radiated with ultrasound radiation and its reflection, is larger because the damage caused to louse 590, e.g., the digestive tract is damaged and the contents are diffused throughout the louse's body. A wounded louse may die immediately or within a short time period after treatment, for example, up to 8 hours. The difference between a louse egg before treatment 594 and a louse egg after treatment 595 may be caused by damage to internal soft tissue of the egg and may lead to liquid content leak out.

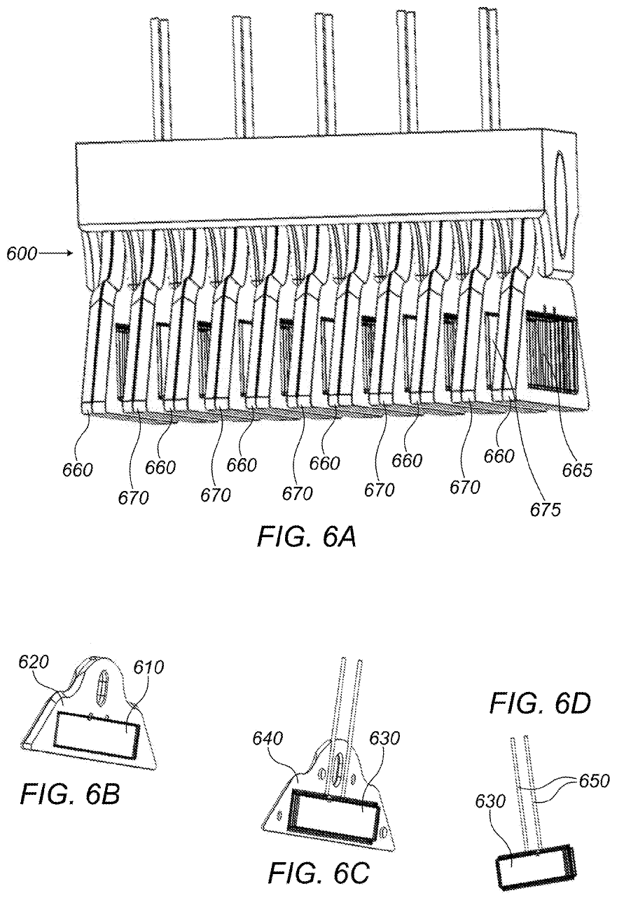

Reference is made now to FIG. 6A, which is a schematic illustration of an exemplary row of teeth of a device for damaging parasites according to some embodiments of the invention. A hand held device, e.g., hand held device 110 of FIG. 1, may include a plurality of teeth extending from a base of the device. The teeth may be arranged in any applicable arrangement, for example, in rows. An exemplary row 600 may include a plurality of piezoelectric teeth or “vibrating teeth” 660 and a plurality of teeth having a reflective element 670, also referred to herein as “reflecting teeth”. Teeth 660 and teeth 670 may be arranged such that each piezoelectric tooth may have one or more reflecting teeth in proximity to it. Such an arrangement may allow ultrasonic radiation generated or transmitted from the vibrating tooth towards the reflecting tooth to be reflected by one or more of the reflecting chambers of one or more reflecting teeth located in proximity to the piezoelectric tooth.

In the exemplary illustration of FIG. 6A a plurality of piezoelectric teeth 660 and a plurality of teeth having a reflective element 670 are arranged in a row 600. The reflecting teeth 670 and piezoelectric teeth 660 are arranged alternately, such that each piezoelectric tooth is placed or located between two reflecting teeth and each reflecting tooth is placed or located between two reflecting teeth. It should be clear that the number of teeth shown in FIG. 6A is an exemplary number and any number of teeth may be used. It should also be clear that row 600 may start either with a reflecting tooth or with a piezoelectric tooth.

In some embodiments of the invention, each reflecting teeth 670 may include or may be made entirely by a reflective material, such as metal sheet or other reflective cover. In other embodiments, each tooth of reflecting teeth 670 may include a reflective chamber, a reflective cavity or a reflective space 675 which may include reflecting material or substance, such as gas, metal and any other material to capable of reflecting ultrasonic radiation. Except for the reflective space 675, each tooth from reflecting teeth 670 may include non-reflective material such as, for example, plastic, which absorbs the acoustic radiation transmitted from a piezoelectric tooth from teeth 660 and does not reach reflective space 675. In some embodiments of the invention, reflective chamber 675 may include a hole, space or area designed to include, contain or hold an elastic balloon as described with reference to FIG. 6B. Elastic balloon 610 may be located inside a frame 620. Frame 620 may be designed such as to fit inside reflective space 675 of each one of teeth 670. Frame 620 may be made from non-reflective material disposed around reflective space 675 such as for example, plastic which absorbs acoustic waves transmitted from teeth 660 that may not reach balloon 610. Balloon 610 may be made from a material which is acoustically transparent with a low acoustic attenuation which does not affect or influence the propagation of ultrasonic radiation, for example, a transparent nylon. and may include air or other gas that may reflect the acoustic radiation transmitted from vibrating teeth 660. Acoustic reflection is created by the differences in acoustic impedance between different materials. As the elastic characteristic of balloon 610, as well as the air included within balloon 610, may allow balloon 610 to act as a spring or coil that may bring a hair or a parasite positioned in proximity to balloon 610 to be in contact or to be in proximity to the neighbor piezoelectric tooth. As the acoustic impedance of liquid as water, gel or a louse is high relative to acoustic impedance of gas, the acoustic radiation may be reflected by the reflecting tooth.

Embodiments of the invention may allow inflating balloon 610 by a predetermined amount of gas until reaching a desired thickness or shape of balloon 610. Balloon 610 may be flat and may have a shape of a rectangle while no gas is inflated into it and may become cylindrical with a predetermined three dimensional characteristics after gas is floated into it. Such an elastic balloon may allow controlling of the actual thickness of each one of teeth 670 and therefore allow approximating a location of a parasite to an adjacent piezoelectric tooth. Balloon 610 may be generated by welding of two elastic rectangle sheets and inflation of balloon 610 may take place once before use of the system for damaging parasites or on scheduled or non-scheduled times during use of the system for damaging parasites.

Reference is made back to FIG. 6A. Each tooth of piezoelectric teeth 660 may include a chamber, a cavity or a space 665 in which piezoelectric material may be positioned. In some embodiments of the invention, chamber 665 may include a hole, space or area designed to include, contain or hold a balloon 630 as described with reference to FIG. 6C. Balloon 630 may be located inside a frame 640. Frame 640 may be designed such as to fit inside space 665 of each one of teeth 660. Frame 640 may be made from two symmetric and/or identical elements designed to match and include balloon 630. A piezoelectric transducer may be located inside balloon 630 in order to allow each one of teeth 660 to vibrate. Balloon 630 may be filled with liquid such as water, ultrasonic gel, fuel or any other liquid. The volume of balloon 630 may be determined and controlled according to the amount of the liquid inserted into it and may allow further controlling of the space between two adjacent teeth.

In some embodiments balloon 630 may be connected to, attached to or coupled to two or more electric conductors, such as electrical wires as shown for example, in FIG. 6D. The electrical wires 650 may be placed within dedicated tubes which may be used for transferring the liquid into balloon 630. In some embodiment of the invention, such tubes may be sealed after the first use. Placing a piezoelectric transducer or piezoelectric material within the liquid in elastic balloon 630 may allow crating uniform, constant and homogeneous load on the piezoelectric material which may enable control of uniform amplitude of ultrasonic radiation. For example, use of balloon 630 may allow uniform amplitude of ultrasonic radiation while a hair is located between two adjacent teeth and when no hair is located between two adjacent teeth.

According to embodiments of the invention, a parasite which is placed between a piezoelectric tooth, e.g., one of teeth 660 and an adjacent reflecting tooth, e.g., the adjacent reflecting tooth of teeth 670, may be radiated with ultrasonic energy from the piezoelectric tooth 660 and with reflected ultrasonic energy arriving from the reflective chamber of the reflecting tooth, e.g., from reflective chamber 675 of tooth 670. In some embodiments of the invention, a parasite which is placed between a piezoelectric tooth and an adjacent reflecting tooth may be radiated with ultrasonic energy from the piezoelectric tooth and with reflected ultrasonic energy arriving from the reflective chambers.

The following example depicts efficiency of a system using ultrasonic radiation for damaging in which reflecting tooth is made of stainless steel. It should in no way be construed, however, as limiting the broad scope of the invention.

EXAMPLE: A reflective tooth made of stainless steel was used in a system for damaging parasites, e.g, a lice comb as presented in embodiments of the invention. Using stainless steel as a reflective material did not decrease lice's mortality rate compared to a reflective tooth comprising gas as a reflective material. When lice were treated with comb having gas as reflective material, the mortality rate (M.R.) was above 85%. Using stainless steel as a reflective material maintained the M.R. higher than 85%, where 73% M.R was reached one hours post activation and 91% M.R was reached three hours post activation. A reflective tooth made of plastic as an absorbing material was used in a system for damaging parasites and reached 24% M.R one hour post activation and 28% M.R three hours post activation.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A device for damaging a parasite while being on a surface of a body of a mammal, comprising:
an ultrasound transmitter for generating ultrasound radiation;
a plurality of teeth extending from a base of the device, wherein the plurality of teeth comprises a vibrating tooth and a reflecting tooth, wherein the ultrasound radiation is transmitted from the vibrating tooth and reflected by the reflecting tooth
to allow damaging a parasite located between the vibrating tooth and the reflecting tooth.

2. The device of claim 1, wherein the reflecting tooth comprises a reflecting material.

3. The device of claim 1, wherein the reflecting tooth comprises a reflecting chamber.

4. The device of claim 3, wherein the reflecting chamber comprises gas.

5. The device of claim 3, wherein the reflecting chamber comprises gas captured within an acoustically transparent material.

6. The device of claim 1, wherein the vibrating tooth vibrates by an ultrasonic source.

7. The device of claim 1, wherein the parasite is hit by the ultrasound radiation that is transmitted from the vibrating tooth towards the reflecting tooth and from at least a portion of the ultrasound radiation reflected by the reflecting tooth.

8. The device of claim 1, wherein the plurality of teeth comprises a plurality of comb teeth.

9. The device of claim 1, wherein the plurality of teeth comprises a plurality of vibrating teeth and a plurality of reflecting teeth arranged alternately.

10. A method for damaging a parasite while being on a surface of a body of a mammal comprising:
placing a handheld device on the surface of the body of the mammal; generating ultrasound radiation;
transmitting the ultrasound radiation from a vibrating tooth towards a reflecting tooth;
reflecting the ultrasound radiation by the reflecting tooth; and
damaging a parasite located between the vibrating tooth and the reflecting tooth by the ultrasound radiation from the vibrating tooth and at least a portion of the ultrasound radiation reflected by the reflecting tooth.

11. The method of claim 10, wherein the reflecting tooth comprises a reflecting material.

12. The method of claim 10, wherein the reflecting tooth comprises a reflecting chamber.

13. The method of claim 12, wherein the reflecting chamber comprises gas.

14. The method of claim 10, wherein the vibrating tooth vibrates by an ultrasonic source.

15. The method of claim 10, wherein the handheld device comprises a plurality of teeth.

16. The device of claim 10, wherein the plurality of teeth comprises a plurality of vibrating teeth and a plurality of reflecting teeth arranged alternately.

17. A device for damaging a parasite on a surface of a body of a mammal, the device comprising:
a base;
at least one ultrasound element configured to generate ultrasound waves; and
a plurality of teeth extending from the base of the device,
wherein the plurality of teeth comprises (a) at least one ultrasound tooth and (b) at least one reflecting tooth, wherein the at least one ultrasound tooth is coupled to one of the at least one ultrasound element, wherein the at least one reflecting tooth is configured to reflect at least a portion of ultrasound waves impacting the reflecting tooth,
wherein the at least one ultrasound tooth and the at least one reflecting tooth are arranged such that the ultrasound waves are transmitted from the at least one ultrasound element and reflected by the at least one reflecting tooth to allow damaging a parasite located between the at least one ultrasound tooth and the at least one reflecting tooth.

18. The device of claim 17, wherein the at least one reflecting tooth comprises a reflecting material.

19. The device of claim 17, wherein the at least one reflecting tooth comprises a reflecting chamber.

20. The device of claim 19, wherein the reflecting chamber contains gas.

21. The device of claim 19, wherein the reflecting chamber contains gas captured within an acoustically transparent material.

22. The device of claim 17, wherein the at least one ultrasound element is configured to cause the at least one vibrating tooth to vibrate.

23. The device of claim 17, wherein the at least one ultrasound tooth and the at least one reflecting tooth are arranged so as to allow damaging a parasite both by the ultrasound waves transmitted from the at least one ultrasound element and by at least a portion of the ultrasound waves reflected by the at least one reflecting tooth.

24. The device of claim 17, wherein at least some of the plurality of teeth are configured to comb hair.

25. The device of claim 17, wherein the plurality of teeth comprises a plurality of vibrating teeth and a plurality of reflecting teeth arranged alternately.

26. The device of claim 17, wherein the ultrasound element includes an ultrasound transducer.

27. The device of claim 17, wherein the ultrasound element includes a piezoelectric element.

28. A method for damaging a parasite on the body of a mammal, the method comprising:
positioning a device in proximity to the body of the mammal, the device comprising:
a base;
at least one ultrasound element configured to generate ultrasound waves; and
a plurality of teeth extending from the base of the device,
wherein the plurality of teeth comprises (a) at least one ultrasound tooth and (b) at least one reflecting tooth, wherein the at least one ultrasound tooth is coupled to one of the at least one ultrasound element, wherein the at least one reflecting tooth is configured to reflect at least a portion of ultrasound waves impacting the reflecting tooth,
wherein the at least one ultrasound tooth and the at least one reflecting tooth are arranged such that the ultrasound waves are transmitted from the at least one ultrasound element and reflected by the at least one reflecting tooth to allow damaging a parasite located between the at least one ultrasound tooth and the at least one reflecting tooth;
operating the device so as to cause the at least one ultrasound element to generate the ultrasound waves, whereby the ultrasound waves damage a parasite located between the at least one ultrasound tooth and the at least one reflecting tooth.

29. The device of claim 28, wherein the at least one reflecting tooth comprises a reflecting material.

30. The device of claim 28, wherein the at least one reflecting tooth comprises a reflecting chamber.

31. The device of claim 30, wherein the reflecting chamber contains gas.

32. The device of claim 30, wherein the reflecting chamber contains gas captured within an acoustically transparent material.

33. The device of claim 28, wherein the at least one ultrasound element is configured to cause the at least one vibrating tooth to vibrate.

34. The device of claim 28, wherein the at least one ultrasound tooth and the at least one reflecting tooth are arranged so as to allow damaging a parasite both by the ultrasound waves transmitted from the at least one ultrasound element and by at least a portion of the ultrasound waves reflected by the at least one reflecting tooth.

35. The device of claim 28, wherein the plurality of teeth comprises a plurality of vibrating teeth and a plurality of reflecting teeth arranged alternately.

36. The device of claim 28, wherein the ultrasound element includes a piezoelectric element.
